(19) United States
(12) Patent Application Publication
(10) Pub. No.: US 2024/0216546 A1
(43) Pub. Date: Jul. 4, 2024

(54) MODEL OPTIMIZED DRUG AND DRUG COMBINATIONS FOR TREATING CANCERS

(71) Applicant: My Personal Therapeutics, London (GB)

(72) Inventors: Santiago Nahuel Villegas, London (GB); Annick Sawala, London (GB); Deisy Guiretti, London (GB); Cristina De Meguel Vijandi, London (GB); Laura Towart, London (GB)

(21) Appl. No.: 18/289,186

(22) PCT Filed: May 3, 2022
(86) PCT No.: PCT/IB2022/000352

§ 371 Date: Nov. 1, 2023

Related U.S. Application Data
(60) Provisional application No. 63/183,915, filed on May 4, 2021.

(51) Int. Cl. A61K 49/00

(52) U.S. Cl. CPC A61K 49/0008

(57) ABSTRACT

The present invention provides for model optimized drugs or combinations of drugs for the treatment of cancers associated with expression at least one human mutant KRAS protein variant, for example oncogenic G12KRAS human variants G12A, G12C, G12D, and G12V. The drugs or combinations of drugs are optimized by using genetically modified Drosophila that express the human mutant KRAS protein variant or an ortholog thereof that promotes tumorigenesis in the Drosophila, and evaluating whether the drug or combination of drugs results in the inhibition of tumorigenesis in the Drosophila.

### FIELD OF THE INVENTION

The invention relates to model optimized drug and drug combinations for treating human cancers associated with KRAS protein variants.

### BACKGROUND

Cancers are attributed to nearly 10 million deaths globally. Mutations in the genes encoding RAS proteins, a family of proteins comprising KRAS, NRAS, and HRAS, are the most frequent genetic mutations resulting in human cancer. KRAS, specifically, is the most frequently mutated driver oncogene in humans, occurring in approximately 30% of all cancers, and emblematic of several notoriously aggressive forms of cancer. For example, over 80% of pancreatic cancers, about 50% of colon cancers, over 35% of colorectal cancers, and over 25% of lung cancers are associated with a mutation of the KRAS gene.

Given their prevalence in cancer, unfortunately, KRAS mutations are also associated with poorer response to most common treatments for cancer. For example, clinical trials have shown a poorer response to chemotherapy measured by a shorter time-to-progression and worse overall survival associated with specific mutations in the KRAS gene. KRAS mutations are also one of the most important predictors of resistance to targeted therapies, including EGFR inhibitors common in treating non-small cell lung cancer and colorectal cancers. For example, in a randomized phase III clinical trial in which the drug erlotinib was administered to patients afflicted non-small cell lung cancer in combination with a first line “gold standard” chemotherapy (carboplatin and paclitaxel), patients with the KRAS mutation exhibited a shorter time to progression (three months) and a shorter overall survival (four months) when treated with the combination compared to the group with wild-type KRAS, for whom the time-to-progression was 12 months. In colorectal cancers, wild-type KRAS identification has become routine clinical practice in determining whether to administer an EGFR inhibitor at all, and the FDA in 2009 updated the labels for the EGFR inhibitors Vectibix and Erbitux to reflect this understanding.

As a result, since its discover in 1982, the KRAS gene itself has been viewed as an attractive drug target for cancers. Unfortunately, however, the KRAS gene lacks obvious binding sites, preventing any significant pharmaceutical development associated with the gene. Recently, several therapies to block KRAS activity are, including anti-sense oligonucleotides, siRNAs, and farnesyltransferase inhibitors (FTIs), have shown some pre-clinical promise in targeting the KRAS gene. However, clinical development has nevertheless stalled. For example, in Phase II and Phase III clinical trials conducted to date, KRAS FTIs were found to likely be insufficient to inhibit the mutated-overactive forms of KRAS protein. Consequently, in nearly four decades, there has been no clinically effective drug developed to target the KRAS gene in treating cancers, to the point that the KRAS gene has been considered an undruggable target.

### SUMMARY

The present invention provides for model optimized drugs or combinations of drugs for the treatment of cancers associated with expression at least one human mutant KRAS protein variant. The drugs or combinations of drugs are optimized by using genetically modified Drosophila that express a human mutant KRAS protein variant or an ortholog thereof that promotes tumorigenesis in the Drosophila, and evaluating whether the drug or combination of drugs results in the inhibition of tumorigenesis in the Drosophila.

The present invention is based on the discovery that, not only the position but also the type of amino acid exchange influences the malignant transforming capacity of mutant KRAS proteins. For example, activating mutations in G12A, G12C, G12D and G12V are highly predominant, and each has a differential drug response, with the type of mutation determinative of the effectiveness of different KRAS inhibitors. KRAS G12C-specific inhibitors, for example, do not work to inhibit KRAS G12D mutations.

Advantageously, the present invention provides strategies for optimizing drugs and drug combinations for inhibiting mutant KRAS that have proved effective in vitro in order to answer the question of whether the drug or drug combination will work in vivo. The present invention provides for the use genetically modified Drosophila that express different variants of a human mutant KRAS protein or an ortholog thereof, for example oncogenic G12 KRAS human variants G12A, G12C, G12D, and G12V, and evaluating whether the drug or combination of drugs results in the inhibition of tumorigenesis in the Drosophila. Advantageously, it has been discovered by the present invention that these variants, when expressed in the fly intestine, may generate tumors that resemble human colorectal malignant growths. Accordingly, the models of the present invention can be used for KRAS variant-specific anti-cancer drug discovery to optimize, screen, and/or validate the response to KRAS specific inhibitors under investigation and development.

Importantly, aspects of the present invention include treating cancer in a human subject with a cancer that is associated with expression of at least one mutant KRAS protein variant. The invention comprises administering a therapeutic regimen comprising a drug or a combination of drugs to a subject that when first provided to genetically modified Drosophila that express the same human mutant KRAS protein variants or an ortholog resulted in the inhibition of tumorigenesis in the Drosophila.

Different missense mutations of the KRAS gene aberrantly activate the KRAS protein, leading to signaling pathway dysregulation and tumorigenesis. The most frequent amino acid substitutions affect the codon 12. KRAS mutations also occur in other codons, including codons 13, 18, 61, 117, and 146, at lower frequencies. Advantageously, in aspects of the present invention, the genetically modified Drosophila may express a human mutant KRAS protein variant is associated with a mutation in at least one of KRAS codon 12, 13, 18, 61, 117, or 146. The at least one human mutant KRAS protein variant may comprise a mutation in at least one of G12A, G12C, G12D, and/or G12V.

In aspects of the invention, the genetically modified Drosophila may express the at least one human mutant KRAS protein variant or an ortholog upon induction through an external factor, for example temperature. Expression of the at least one human mutant KRAS protein variant or ortholog may inhibit the Drosophila larva from surviving to pupation. Because the drug or combination of drugs, when given to the Drosophila larva may allow the Drosophila larva to survive to pupation, the drug response may be quickly and visibly identified.

The present invention may allow for treatment of cancers including adenocarcinoma of the lung and/or colon. The therapeutic regimen administered to the subject may comprise an anti-cancer therapy, for example a known anti-cancer therapy. The anti-cancer therapy may be selected from the group consisting of alkylating agents, anthracyclines, cytoskeletal disruptors, histone deacetylase inhibitors, inhibitors of topoisomerase I, inhibitors of topoisomerase II, kinase inhibitors, nucleotide analogs and precursors analogs, peptide antibiotics, platinum-based agents, retinoids, and vina aklaloids, and derivatives thereof.

Aspects of the invention also include a method for screening drugs or a combination of drugs for the treatment of a cancer associated with expression at least one human mutant KRAS protein variant. Methods comprise providing the drug or combination of drugs to genetically modified Drosophila that express the at least one human mutant KRAS protein variant or an ortholog and promotes tumorigenesis in the Drosophila, and evaluating whether the drug or combination of drugs results in the inhibition of tumorigenesis in the Drosophila.

Advantageously, in aspects of the present invention, the genetically modified Drosophila may express a human mutant KRAS protein variant is associated with a mutation in at least one of KRAS codon 12, 13, 18, 61, 117, or 146. The at least one human mutant KRAS protein variant may comprise a mutation in at least one of G12A, G12C, G12D, and/or G12V.

In aspects of the invention, the genetically modified Drosophila may express the at least one human mutant KRAS protein variant or an ortholog upon induction through an external factor, for example temperature. Expression of the at least one human mutant KRAS protein variant or ortholog may inhibit the Drosophila larva from surviving to pupation, and the drug or combination of drugs may be provided to the Drosophila larva. Because the drug or combination of drugs, when given to the Drosophila larva may allow the Drosophila larva to survive to pupation, the drug response may be quickly and visibly identified by evaluating whether the Drosophila larva survive to pupation.

The present invention may allow for treatment of cancers including adenocarcinoma of the lung and/or colon. The therapeutic regimen administered to the subject may comprise an anti-cancer therapy, for example a known anti-cancer therapy. The anti-cancer therapy may be selected from the group consisting of alkylating agents, anthracyclines, cytoskeletal disruptors, histone deacetylase inhibitors, inhibitors of topoisomerase I, inhibitors of topoisomerase II, kinase inhibitors, nucleotide analogs and precursors analogs, peptide antibiotics, platinum-based agents, retinoids, and vina aklaloids, and derivatives thereof.

### DETAILED DESCRIPTION

The present invention provides for model optimized drugs or combinations of drugs for the treatment of cancers associated with expression at least one human mutant KRAS protein variant. The drugs or combinations of drugs are optimized by using genetically modified Drosophila that express a human mutant KRAS protein variant or an ortholog thereof that promotes tumorigenesis in the Drosophila, and evaluating whether the drug or combination of drugs results in the inhibition of tumorigenesis in the Drosophila.

Prior to the present invention, genetic techniques available in the Drosophila have been limited. Although Drosophila have a very rapid life cycle that allows for the production of transgenic lines quickly and Drosophila organs perform the equivalent functions of the mammalian heart, lung, kidney, gut and reproductive tract, as well as a sophisticated brain and visual system, transgenic Drosophila lack certain features that limit its use as a cancer model. For example, Drosophila lack an adaptive immune system that humans possess for developing immunological memory involved in checkpoints that limit tumor growth. Additionally, Drosophila do not have a vascular circulatory system, which limits the study of factors affecting tumor angiogenesis and growth. Accordingly, Drosophila models have not been available as a model for the screening of drug or drug candidates for the treatment of cancers associated with KRAS gene mutations. Vertebrate animal models are also limited. For example, human tumor xenografts injected into mice do not exhibit tumorigenesis or metastasis in the subject, and the mice also lack the adaptive immune system that plays a role in controlling tumor growth. Moreover, genetically engineering mice is time consuming and difficult to do for the multiple mutations involved in a subject's cancer.

The present invention provides for the improved use of Drosophila models for the screening of drugs or drug combinations for the treatment of cancers associated with mutations in the KRAS gene. The present invention is based on the discovery that, not only the position but also the type of amino acid exchange influences the malignant transforming capacity of mutant KRAS proteins. For example, activating mutations in G12A, G12C, G12D and G12V are highly predominant, and each has a differential drug response, with the type of mutation determinative of the effectiveness of different KRAS inhibitors. KRAS G12C-specific inhibitors, for example, do not work to inhibit KRAS G12D mutations.

Accordingly, aspects of the present invention provide for methods of treating cancer in a human subject afflicted with a cancer associated with expression of at least one human mutant KRAS protein variant comprising administering to the subject a drug or combination of drugs. Advantageously, the drug or combination of drugs, when provided to genetically modified Drosophila that express at least one human mutant KRAS protein variant or an ortholog thereof that promotes tumorigenesis in the Drosophila, the drug results in the inhibition of tumorigenesis in the Drosophila.

In aspects of the invention, multiple Drosophila, reflecting different KRAS mutations in a population of patients diagnosed with cancer can be used to screen and select therapeutic regimens for treatment and to screen for new drug or drug combinations and identify new therapeutics for inhibiting or treating KRAS-associated cancers. For example, a Drosophila or combination of Drosophila that most closely resemble a patient's KRAS mutation profile may be selected and used to screen and identify therapeutic regimens for the patient. For example, tumors or cancer cells from a human subject may be analyzed to characterize the patient's KRAS mutations. This information may be used to design and construct a Drosophila that expresses the subject's phenotype. In aspects of the invention, the Drosophila is a member of the species Drosophila melanogaster. Methods for genetically modifying Drosophila are described in PCT international patent application publication WO 2017/117344, the entirety of the contents of which are incorporated herein.

In aspects of the invention, the genetically modified Drosophila may express the at least one human mutant KRAS protein variant or an ortholog upon induction through an external factor, for example temperature. Embryos may be placed on their food, and once they begin consuming the food as larvae, the activity of the transgenic orthologs may be induced. As a result, therapeutic efficacy of the regimen may be quickly indicated by survival of the larva. Advantageously, such an assay does not require tumor visualization, sequencing equipment, or detection of markers not compatible with high through-put screening or read-outs.

In aspects of the invention, the cancer to be treated is a pancreatic cancer, colon cancer, colorectal cancers, or lung cancer are associated with a mutation of the KRAS gene. However, the cancer may be any cancer associated with a KRAS mutation. For example, the cancer may be a bladder cancer, bone cancer, breast cancer, cervical cancer, colorectal cancer, esophageal cancer, gallbladder cancer, head and neck cancer, kidney cancer, lung cancer, ocular cancer, oral cancer, ovarian cancer, pancreatic cancer, pelvic cancer, penile cancer, prostate cancer, skin cancer, throat cancer, thyroid cancer, such as medullary thyroid cancer, or uterine cancer.

In aspects of the invention, multiple KRAS mutations may be expressed in the Drosophila. For example, one, two, three, four, five, six, seven, eight, nine, ten or more orthologs of the KRAS oncogene may be is overexpressed in the Drosophila. In aspects of the invention, the activity of one or more of the oncogenes may be increased by at least 10%, 20%, 30%, 40%, 50%, 60%, 70%, 80%, 90%, 100%, 125%, 150%, 175%, 200%, 250%, or 300% or more as compared to the activity of the one or more oncogenes in a non-modified Drosophila.

In aspects of the invention, the activity of the one or more oncogenes may be increased by genetically engineering the Drosophila to express cDNAs encoding the one or more orthologs, which expression of the cDNAs are regulated by exposure to the external factor. In aspects of the invention, the activity of the one or more tumor suppressing genes may be inhibited by genetically engineering the Drosophila to express interfering RNAs that interfere with expression or translation of the one or more tumor suppressor inhibitors.

In aspects of the invention, the drug or combination of drugs may be provided to a subject that has failed standard of care therapy. In aspects of the invention, there may be no standard of care therapy for the cancer.

A combination of drugs means the use of more than one drug. The two or more drugs may be administered in one or more dosage forms. The combination of drugs may be administered to the subject in any order. For example, a first therapy can be administered prior to, concomitantly with, or subsequent to the administration of a second therapy to a subject.

In aspects of the invention, the Drosophila models of the present invention may be used to evaluate any therapy. A therapy means any protocol, method, and/or agent that can be used in the treatment of cancer. A therapy may be a chemotherapy, immune modulatory drugs, and/or other therapies useful in the treatment of a cancer. The therapy may include an adjuvant therapy. For example, a therapy in conjunction with a drug therapy, biological therapy, hormonal therapy, surgery, and/or supportive therapy.

Treating means administration of a therapy from which a subject receives a beneficial effect, for example as the reduction, decrease, attenuation, diminishment, stabilization, remission, suppression, inhibition or arrest of the development or progression of cancer, or a symptom thereof. The treatment that the subject receives may not cure the cancer, but may prevent the progression or worsening of the cancer. In aspects of the invention, the treatment may not prevent the onset of cancer, but may prevent the onset of cancer symptoms. Advantageously, aspects of the present invention include the treatment of cancers associated with KRAS, previously resistant to known therapies.

Methods well known to those skilled in the art can be used for a genomic, proteomic, or phenomic analysis of a samples obtained from a subject suffering from a cancer associated with a KRAS mutation. Deep sequencing techniques are advantageous in capturing the full repertoire of KRAS mutations, and to ensure that the identification of mutations in tumor subpopulations is captured. For example, biological material may be removed from a patient suffering from a cancer associated with KRAS, preferably a sample comprising the patient's cancer cells or a portion of the patient's tumor. Genomic analysis of the patient sample may be conducted utilizing techniques known to one of skill in the art. For example sequence analysis of a patient sample may include DNA sequencing, RNA sequencing, first-generation sequencing, next-generation sequencing, massively parallel sequencing, whole genome sequencing, whole exome sequencing and single cell sequencing.

Proteome analysis may be used to analyze protein expression in the sample. For example, mass spectrometry, including tandem mass spectrometry, high-resolution mass spectrometry, high-throughput mass spectrometry, and fluorescence imaging-based methods may be used to perform proteome analysis.

Aspects of the invention comprise identifying or expression orthologs of human KRAS mutations that may be expressed in Drosophila. A computational assessment may be used to identify an ortholog of the KRAS mutation in Drosophila. Identifying the ortholog may comprise identifying an ortholog that can be manipulated to recapitulate the activities found in a population of subjects suffering from cancer associated with the same KRAS mutation. The functional similarity of a gene or protein in the organism to the KRAS mutation may be determined by gene ontology (GO), IntelliGO, and EnzDP. The functional similarity of the KRAS mutation in the organism to the disease driver may be determined by any method known to one of skill in the art. For example, one or more databases may be used to identify an ortholog such as Flybase, Homologene, TreeFam, OrthoMCI, MetaPhOrs, P-POD (Princeton Protein Orthology Database), KEGG Orthology, EggNog, POGS (plant-specific), OrthoDB, Roundup, ProtozoaDB, and Gene-Oriented Orthology Database.

Drosophila of the present invention may be modified to overexpress an ortholog of a KRAS mutation or may be modified to underexpress an ortholog of KRAS mutation. Altering the expression or activity of the KRAS mutation in the organism may result in a lethal phenotype or near-lethal phenotype. Transgenes may be used to modulate the expression of the kRAS mutation. The transgene may comprise a cDNA of the ortholog the KRAS mutation, a cDNA of an activator of the KRAS mutation, a cDNA of an inhibitor of the KRAS mutation, an interfering RNA, including small interfering RNA or short hairpin RNA, that targets the KRAS mutation, or a combination thereof.

To model reduced activity of multiple KRAS tumor suppressors, a series of interfering RNAs may be designed to reduce activity of the KRAS tumor suppressor, and may be fused together by standard cloning methods. The resulting oligonucleotide can then be fused 3′ to a promoter, for example an inducible promoter element or upstream activating system. The single fused genetic element may silence as many as ten or more genes. The genetic element may be cloned into a multiple cloning site of a plasmid. The genetic element may be cloned into a remaining multiple cloning site within a plasmid carrying an over-expressed KRAS mutation.

To elevate activity of Drosophila KRAS orthologs, a transgene representing an ortholog of each of the patient's KRAS mutation may be fused to a promoter and cloned into a multiple cloning site contained within a transformation plasmid. The transgene may be the unaltered sequence or can contain activating mutations that model activation of the human ortholog within the patient's tumor. The transgene may be an unaltered sequence or an ortholog contain an activating mutation that models the activation of the human ortholog. The ortholog may be modified by introducing a mutation in the ortholog that alters protein function to recapitulate the patient's KRAS phenotype.

The transgene may be introduced in a coding region, promoter, termination sequence, and/or enhancer region of a KRAS gene. The mutation may occur in an exon, intron, or untranslated region of the KRAS gene. The one or more mutations in the KRAS gene may be a deletion, an insertion, a substitution, and/or a frameshift mutation, to name a few. Alternatively, where inhibition of activity of the KRAS mutation desired interfering RNA silencers may be used.

In aspects of the invention, the expression of the transgene is controlled and regulated. Inducible control of the transgene within the transgenic Drosophila model can be used to ensure that the engineered ortholog is not expressed until the Drosophila founder lines are established and the larvae is ready to be fed the drug or combination of drugs. An inducible promoter that is temperature sensitive may be used to regulate transgene expression so that an increase in culture temperature can be used to turn on the transgene, which in turn modulates the expression of the KRAS mutation. For example, when expression of the transgene is regulated in a temperature sensitive manner, the transgene may be expressed when the Drosophila is cultured at a certain temperature of 20° C. to 29º C but not at a temperature of 18° C.

The transgene may be linked to a promoter regulated by a transcriptional activator, and the activator may be linked to a temperature sensitive promoter so that only when expression of the transcriptional activator is induced at a certain temperature is the transgene expressed. The transgene may be linked to a promoter regulated by a transcriptional repressor and the repressor may be linked to a temperature sensitive promoter so that only when expression of the repressor is inhibited at a certain temperature is the transgene expressed. Expression of the transgene result in a change in the genomic profile, proteomic profile, and/or phenomic profile of the Drosophila as compared to non-modified organism.

In aspects of the invention, expression of a transgene should be engineered to occur in an organ vital to the survival of the Drosophila. The expression of a transgene in an organ vital to survival of the Drosophila enables high throughput, rapid read out—rescue from lethality upon induction of the transgene. Examples of vital organs of the Drosophila include, but are not limited to, the hindgut, midgut imaginal disc, nervous system, fat body, nephrocyte, heart, trachea, thorax, abdomen, and malphigian tubules. Expression of the transgene may also occur outside an organ vital to the survival of the organism and in another cell type, tissue or organ of the organism. For example, expression of the transgene may occur in the hindgut, wing, ovaries, genitalia, or eye of the organism. Advantageously, it has been discovered by the present invention that these variants, when expressed in the Drosophila intestine, may generate tumors that resemble human colorectal malignant growths.

In aspects of the invention, the transgene is regulated, directly or indirectly, by an inducible promoter such that when expression of the transgene in an organ vital to survival is induced it results in 80%, 85%, 86%, 87%, 88%, 89%, 90%, 91%, 92%, 93%, 94%, 95%, 96%, 97%, 98%, 99% or 100% lethality of the pupa or the larvae stage prior to pupariation, for example stage 4 of the larvae. and 98%, 99% or 100% lethality of adult flies.

Constructs, for example DNA plasmids, comprising a transgene may be used to alter the expression level or activity of an ortholog of a KRAS gene in the. The construct may comprise one or more recombinase recognition sites. The two or more recombinase recognition sites may flank one or more RNAis, one or more cDNAs, or both. The recombinase recognition site may be an attP site. The construct for altering the expression level or activity of an ortholog of KRAS in the Drosophila may be a DNA plasmid and a transgene is inserted into a region between two inverted repeat elements on the DNA plasmid. The inverted repeat element may be a P element. Alternatively, the transgene may be inserted into a region between two recombination sites into a construct for altering the expression level or activity of the KRAS gene in the Drosophila. The construct for altering the expression level or activity of an ortholog of a KRAS mutation in the Drosophila may comprise a flipase (Flp)-out cassette. A Flp-out cassette may comprise two flipase recognition target sequences (FRTs). The construct may further comprise a transgene located between the two FRTs. The transgene may be flanked by FRT sites, which results in silencing of the transgene. Expression of the transgene can occur by excision of the FRT sites by flipase (Flp). The construct may comprise a Flp-out cassette placed between a promoter and a transgene. The construct may comprise a OC31-integrase mediated cassette and a transgene. The OC31-integrase mediated cassette comprises two attP sites. The transgene may be located between the two attP sites. Exemplary expression systems that can be used for introducing a transgene into the Drosophila include GAL4-UAS, LexA-/exAop, and/or QF-QUAS systems.

The expression system that can be used for introducing a transgene into the Drosophila may be a binary expression system. The binary expression system may comprise a transcriptional activator and a transgene under the control of a promoter that is under the control of the transcriptional activator. The transcriptional activator may be expressed in a specific pattern. Alternatively, the transcriptional activator may be randomly expressed. The expression system may comprise a ternary expression system. The ternary expression system may comprise a transcriptional activator, a transgene under the control of a promoter, and a repressor of the transcriptional activator. The promoter that controls the transgene may be under the control of the transcriptional activator. Examples of ternary expression systems include, but are not limited to, GAL80, split Gal4 and split LexA. The expression system may comprise a modular expression system. The modular expression system may comprise a convertible genetic platform. Generally, a modular expression system uses multiple recombinase systems to swap a transcriptional activator sequence with another sequence. For example, the integrase swappable in vivo targeting element (InSITE) system uses three different site-specific recombinases (e.g., Flp-FRT, Cre recombinase-Lox, and OC31 integrase-attP/attB) to swap GAL4 with another sequence.

The expression system may comprise one or more inducible promoters. The expression systems may comprise one or more cell- or tissue-specific promoters. Examples of cell- or tissue-specific promoters include, but are not limited to, GMR promotor, Byn promoter, Btl promoter, Eyeless promoter, 765 promoter, Sd promoter, Ptc promoter, Omb promoter, B29 promoter, CD 14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, mouse INF-β promoter, Mb promoter, nphsl promoter, OG-2 promoter, SP-B prmoter, SYN1 promoter, WASP promoter, SV40/bAlb promoter, SV40/hAlb promoter, SV40/CD43 promoter, SV40/CD45 promoter, and NSE/RU5′ promoter. The expression systems may comprise a heat shock promoter, GAL4/UAS system, GAL4/GAL80/UAS system, EGUF system, FLP/FRT-regulated system, QUAS system, and/or lexA system.

The construct or expression system may be temporally expressed in the Drosophila. Temporal expression of the construct or expression system may comprise expression of the construct or expression system during a specific stage of development. For example, the construct or expression system may be expressed during the adult stage of development of the Drosophila. Alternatively, the construct or expression system may be expressed during the pupal or larval stage of development of the Drosophila. In another example, the construct or expression system may be expressed during the embryonic stage of development of the Drosophila.

Advantageously, expression of the transgene may be controlled at will. For example, expression of the transgene may be under the control of an inducible promoter. The inducible promoter may confine expression of the transgene to a specific cell type, tissue or organ, to a specific time, to a specific temperature and/or to a specific light condition. Expression of the transgene can then be induced at will in any cell type, tissue, or organ, at a specific time (e.g., stage of development), at any time, for example in response to a change in temperature or light induced by the operator.

Cloning techniques known to one of skill in the art may be used to clone a cDNA or RNAi into a construct, such as a plasmid. Methods for cloning a cDNA and/or RNAi molecule into a plasmid may comprise inserting the cDNA and/or RNAi molecule into the multiple cloning site (MCS) of a plasmid. Cloning the cDNA and/or RNAi molecule into the plasmid may comprise flanking the cDNA and/or RNAi molecule with inverted repeat elements or recombinase recognition sites. The inverted repeat elements or recombinase recognition sites may be fused to the cDNA and/or RNAi molecule prior to insertion into the plasmid. Alternatively, the plasmid may contain the inverted repeat elements or recombinase recognition sites and the cDNA and/or RNAi molecule may be inserted between the inverted repeat elements or recombinase recognition sites.

Techniques known to one of skill in the art can be used to introduce a construct or expression system into a Drosophila Methods for introducing a construct or expression system into an organism include, but are not limited to transfection, injection (e.g., microinjection), and viral infection. The construct or expression system can be introduced into the Drosophila by injection. For example, the construct and/or expression system can be injected into the posterior germ cells of fly embryos.

In aspects of the invention, the construct or expression system is targeted to a specific location within the organism Drosophila via directed integration. The integration of the construct/expression system into a standard position on a chromosome allows comparison of separate transgenic lines. Expression levels of transgenes and combinations can be confirmed using techniques known to one skilled in the art, e.g., by qPCR and/or fluorescence microscopy (e.g., UAS-GFP).

Genetic engineering techniques known to one of skill in the art can be used to produce the transgenes and constructs described herein as well as Drosophila as described herein, for example genetic engineering techniques utilizing CRISPR RNA-Guided Cas9 Nucleases.

Generating the genetically modified Drosophila may comprise conducting multiple crosses with multiple founder lines to produce the Drosophila whose genomic, proteomic and/or phenomic profile resembles the genomic, proteomic and/or phenomic profile the KRAS mutation, wherein the resemblance of the genomic, proteomic and/or phenomic profiles is based on the similarity of the expression or activity of one or more orthologs of the KRAS mutation in the genomic, proteomic and/or phenomic profile of the Drosophila to the expression or activity of the KRAS mutation in the genomic, proteomic and/or phenomic profile of the disease. A founder line may comprise a Drosophila comprising a transgene that alters the expression or activity of an ortholog of at least one KRAS mutation. A founder line may comprise an organism comprising a transgene that alters the expression or activity of one or more orthologs of one or more KRAS mutations. A founder line may comprise an organism comprising a set of transgenes that alters the expression or activity of one or more orthologs of one or more KRAS mutations.

Generating a Drosophila may comprise: (a) transforming a first Drosophila with a first construct or expression system to produce a first founder line; (b) transforming a second Drosophila with a second construct or expression system to produce a second founder line; and (c) crossing the first Drosophila with the second Drosophila to produce a progeny line. The first Drosophila and/or second Drosophila may be transformed with a construct or expression system of the invention.

Advantageously, the genetically modified Drosophila are useful in high-throughput screens to identify drugs that are beneficial for the treatment of cancer. Once the Drosophila is calibrated to the screenable phenotype of lethality, the Drosophila may be used to screen for drugs that increase the viability of the Drosophila to a certain stage of development. The screening assay may involve inducing expression of the construct or expression system in the Drosophila in the presence or absence of one or more candidate drugs or a combination of candidate drugs; and determining the phenotype of the Drosophila in the presence of the one or more candidate drugs. The Drosophila may be exposed to the candidate drug or combination of candidate drugs by the addition of the candidate drug or combination of candidate drugs to the food or water that is given to the Drosophila. Alternatively, the Drosophila may be exposed to the candidate drug or combination of candidate drugs by injection, such as intravenous injection or peritoneal injection. The amount of drug each Drosophila is exposed to may not be lethal to the non-modified counterpart of the Drosophila. Multiple doses of the same drug or a combination of drugs may be tested in the Drosophila. Certain doses of the drug or a combination of drugs may be more effective in the Drosophila than others. The screening assay may also include exposing the Drosophila to a control composition, such a negative control). For example, the screening assay may comprise placing media comprising food and a control composition into a container or well of the plate with the Drosophila.

The Drosophila may be exposed to the candidate drugs in combinations. A Drosophila may be exposed to two or more candidate drugs at the same time, or sequentially. For example, the Drosophila may be exposed to a first candidate drug and a second candidate drug at a first time point and exposed to a third candidate drug at a second time point. The Drosophila may be exposed to two or more candidate drugs at two or more overlapping time periods. For example, the Drosophila may be exposed to a first candidate drug for a first time period and exposed to a second candidate drug for a second period, wherein the first and second time periods at least partially overlap. The Drosophila may be exposed to two or more candidate drugs at two or more non-overlapping time periods. For example, the Drosophila may be exposed to a first candidate drug for a first time period and exposed to a second candidate drug for a second period, wherein the first and second time periods do not overlap.

The screening assays for drugs for treating cancers associated with KRAS in Drosophila may be performed in individual tubes or wells of plate. Food may be placed into each tube or well. Each candidate drug may be added into duplicate tubes or wells at a chosen concentration that is not lethal to non-modified organism Drosophila. For example, the final food concentration of each candidate drug may be 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 110 μM, 125 μM, 150 μM, 175 μM, or 200 μM. A candidate drug may be mixed into the food and may be allowed to further diffuse for a period of time. A designated number of transgenic embryos may be placed into each tube or well on top of the solidified food/drug mixture. For example, each tube or well may contain 3, 4, 5, 6, 7, 8, 9, 10, 11, 12, 13, 14, 15 or more embryos. Each tube or plate may then be covered with a breathable substance and animals develop at the optimized temperature.

A candidate drug may be determined to be effective if the presence of the candidate drug at least partly rescues the lethality caused by expression of the construct or expression system. For example, a candidate drug may be determined to be effective if it rescues at least 0.5%, 1%, 5%, 10%, 15%, 20%, 25% 30%, 35%, 40%, 45%, 50%, 55%, 60%, 65%, 70%, 75%, 80%, 85%, 90%), 95% or more of Drosophila to adulthood. A candidate drug may be determined to be effective if the presence of the candidate drug reduces the severity of a phenotype caused by expression of the construct or expression system. The candidate drug may be determined to be effective if the candidate drug causes the proteomic and/or phenomic profile of the Drosophila to more closely resemble the proteomic and/or phenomic profile of a healthy subject as compared to the proteomic and/or phenomic profile of the Drosophila in the absence of the candidate drug.

A combination of candidate drugs may be determined to be effective if the presence of the combination of candidate drugs rescues the lethality caused by expression of the construct or expression system. The combination of candidate drugs may be determined to be effective if the combination of candidate drugs causes the proteomic and/or phenomic profile of the Drosophila to more closely resemble the proteomic and/or phenomic profile of a healthy subject as compared to the effect of the separate candidate drugs on the proteomic and/or phenomic profile of the Drosophila. For instance, a combination of candidate drugs comprising drug A, drug B, and drug C may be determined to be effective if the combination of candidate drugs causes the proteomic and/or phenomic profile of the Drosophila to more closely resemble the proteomic and/or phenomic profile of a non-modified Drosophila as compared to the separate effect of drug A, drug B, and drug C on the proteomic and/or phenomic profile of the Drosophila. The combination of candidate drugs may be determined to be effective if the synergistic effect of the combination of candidate drugs on the proteomic and/or phenomic profile of the Drosophila is greater than the effect of the separate candidate drugs on the proteomic and/or phenomic profile of the Drosophila.

Importantly, aspects of the present invention include treating cancer in a human subject with a cancer that is associated with expression of at least one mutant KRAS protein variant. The invention comprises administering a therapeutic regimen comprising a drug or a combination of drugs to a subject that when first provided to genetically modified Drosophila that express the same human mutant KRAS protein variants or an ortholog resulted in the inhibition of tumorigenesis in the Drosophila.

A drug or a combination of drugs may be administered to a cancer patient by any route known to one of skill in the art. For example, the drug or combination of drugs may be administered locally or systemically to a cancer patient. The drug or combination of drugs may be administered to a cancer patient parenterally, orally, topically, transdermally, intranasally, intraperitoneally, intratumorally, intradermally, or intracerebrally. The drug or a combination of drugs may be administered subcuteanously, intravenously, or intramuscularly. If the drug or a combination of drugs is already FDA approved or approved by another regulatory authority, the drug or combination of drugs may be administered by a route previously approved by the FDA or another regulatory authority.

If a combination of drugs is administered to a cancer patient, the drugs may be administered concurrently or sequentially. For example, one drug may be administered prior to (e.g., 5 minutes, 15 minutes, 30 minutes, 45 minutes, 1 hour, 2 hours, 3 hours, 4 hours, 6 hours, 12 hours, 18 hours, 24 hours, 2 days, 5 days, 7 days, 14 days or 21 days prior to) the administration of another drug. The combination of drugs may be administered via the same route of administration or a different route of administration to the cancer patient.

The amount of a drug or a combination of drugs to use to treat a cancer patient may be extrapolated by one of skill in the art from the Drosophila. Alternatively or in addition, the amount of a drug or a combination of drugs to administer to a cancer patient may be determined based on cell culture studies using patient cancer cells exposed to the drug or combination of drugs and/or animal model studies using the drug or combination of drugs. If the drug or a combination of drugs has been FDA approved or approved by another regulatory authority, the dose and/or frequency of administration previously approved can be used to determine the dosing regimen for the drug or combination of drugs. A low dose of a drug or a combination of drugs is initially administered to a cancer patient and the dose is increased over time. In addition, the frequency of the drug or the combination of drugs administered to the patient may be altered. The dose and/or frequency of administration of a drug or a combination of drugs to a cancer patient may be adjusted over time depending on the condition of the patient.

The dose and/or frequency of administration of a drug or a combination of drugs to a cancer patient is adjusted based on the expression or activity of a KRAS mutation in a sample from the subject after administration of the drug or combination or drugs to the subject. For example, the dosing frequency of a drug or a combination of drugs may be increased if the expression or activity of the KRAS mutation is increased in a sample from the patient after administration of the drug or combination of drugs to the cancer patient as compared to the expression or activity of the KRAS mutation prior to administration of the drug or combination of drugs to the cancer patient. The dosing frequency of a drug or a combination of drugs may be increased if the expression or activity of a the KRAS mutation is decreased in the sample from the patient after administration of the drug or combination of drugs to the cancer patient to the patient as compared to the expression or activity of the KRAS mutation prior to administration of the drug or combination of drugs to the cancer patient.

The efficacy of a drug or a combination of drugs for the treatment of a cancer patient may be assessed by measuring one or more biomarkers of a particular cancer. Biomarkers for different types of cancers are known in the art and can be used to assess the efficacy of a drug or a combination of drugs to treat a particular type of cancer in a patient. Techniques known to one of skill in the art can be used to measure the biomarkers at the RNA and/or protein level.

The drug or combination of drugs identified or confirmed in a Drosophila screening assay described by the invention may be tested in one or more other assays prior to providing a list of the results from the screening assay to a healthcare provider and/or the subject; a recommendation for treatment of the subject with a particular drug or combination of drugs; or treating the subject with a particular drug or combination of drugs. The other assays may include assessing the efficacy of the drug or combination of drugs identified in the Drosophila screening assay described by the invention on cancer cells from the subject or a cell line derived from the subject's cancer, evaluating the safety of the drug or combination of drugs identified in the Drosophila screening assay described by the invention on non-cancerous cells, and testing the safety and/or efficacy of the drug or combination of drugs in an animal model of the subject's cancer. If the drug or combination of drugs identified or confirmed in the Drosophila screening assay described by the invention is already FDA approved, there may be no need to conduct any other assays aside from the Drosophila assay to assess the safety of the drug or combination of drugs and/or to assess the therapeutic indices of the drug or combination of drugs.

Method of treating cancer may result in one, two, three or more of the following effects: complete response, partial response, increase in overall survival, increase in disease free survival, increase in objective response rate, increase in time to progression, increase in progression-free survival, increase in time-to-treatment failure, and improvement or elimination of one or more symptoms of cancer. A method of treating cancer may increase overall survival. The method of treating cancer may result in an increase in progression-free survival.

Several endpoints may be used on tumor assessments. These endpoints include disease free survival, objective response rate, time to progression, progression-free survival, and time-to-treatment failure. The collection and analysis of data on these time-dependent endpoints are based on indirect assessments, calculations, and estimates.

Disease free survival is the time from randomization until recurrence of tumor or death from any cause. Although overall survival is a conventional endpoint for most adjuvant settings, disease free survival can be an important endpoint in situations where survival may be prolonged, making a survival endpoint impractical. Disease free survival can be a surrogate for clinical benefit or it can provide direct evidence of clinical benefit. This determination is based on the magnitude of the effect, its risk-benefit relationship, and the disease setting. The definition of disease free survival can be complicated, particularly when deaths are noted without prior tumor progression documentation. These events can be scored either as disease recurrences or as censored events. Although all methods for statistical analysis of deaths have some limitations, considering all deaths (deaths from all causes) as recurrences can minimize bias. Disease free survival can be overestimated using this definition, especially in patients who die after a long period without observation. Bias can be introduced if the frequency of long-term follow-up visits is dissimilar between the study arms or if dropouts are not random because of toxicity.

Objective response rate is the proportion of patients with tumor size reduction of a predefined amount and for a minimum time period. Response duration usually is measured from the time of initial response until documented tumor progression. Generally, the FDA has defined objective response rate as the sum of partial responses plus complete responses. When defined in this manner, objective response rate is a direct measure of drug antitumor activity, which can be evaluated in a single-arm study. If available, standardized criteria should be used to ascertain response. The significance of objective response rate is assessed by its magnitude and duration, and the percentage of complete responses (no detectable evidence of tumor).

Time to progression and progression-free survival are primary endpoints for drug approval. Time to progression is defined as the time from randomization until objective tumor progression; time to progression does not include deaths. Progression-free survival is defined as the time from randomization until objective tumor progression or death. Compared with time to progression, progression-free survival is the preferred regulatory endpoint. Progression-free survival includes deaths and thus can be a better correlate to overall survival. Progression-free survival assumes patient deaths are randomly related to tumor progression. However, in situations where the majority of deaths are unrelated to cancer, time to progression can be an acceptable endpoint.

As an endpoint to support drug approval, progression-free survival can reflect tumor growth and be assessed before the determination of a survival benefit. Its determination is not confounded by subsequent therapy. For a given sample size, the magnitude of effect on progression-free survival can be larger than the effect on overall survival. However, the formal validation of progression-free survival as a surrogate for survival for the many different malignancies that exist can be difficult. Data are sometimes insufficient to allow a robust evaluation of the correlation between effects on survival and progression-free survival. Cancer trials are often small, and proven survival benefits of existing drugs are generally modest. The role of progression-free survival as an endpoint to support licensing approval varies in different cancer settings. Whether an improvement in progression-free survival represents a direct clinical benefit or a surrogate for clinical benefit depends on the magnitude of the effect and the risk-benefit of the new treatment compared to available therapies.

Time-to-treatment failure is a composite endpoint measuring time from randomization to discontinuation of treatment for any reason, including disease progression, treatment toxicity, and death. TTF is not recommended as a regulatory endpoint for drug approval. Time-to-treatment failure may not adequately distinguish efficacy from these additional variables. A regulatory endpoint should clearly distinguish the efficacy of the drug from toxicity, patient or physician withdrawal, or patient intolerance.

Treating a subject may comprise an improvement in and/or the elimination of one or more symptoms of cancer in the subject. Treating a subject comprises an improvement in and/or the elimination of one or more symptoms of colorectal cancer in the subject. The one or more symptoms of colorectal cancer may include changes in bowel habits, constipation, diarrhea, alternating diarrhea and constipation, rectal bleeding or blood in stool, abdominal bloating, abdominal cramps, abdominal discomfort, gas pains, feeling of incomplete bowel emptying, thinner than normal stools, unexplained weight loss, unexplained loss of appetite, nausea, vomiting, anemia, jaundice, weakness, and fatigue or tiredness. Colorectal cancer symptoms may also include pain, fracture, constipation, decreased alertness, shortness of breath, difficulty breathing, coughing, chest wall pain, extreme fatigue, increased abdominal girth, swelling of the feet and hands, yellowing or itch skin, bloating, swollen belly, pain, confusion, memory loss, headache, blurred or double vision, difficulty with speech, difficulty with movement, and seizures.

Treating a subject may comprise an improvement in and/or the elimination of one or more symptoms of lung cancer in the subject. The one or more symptoms of lung cancer may include, but are not limited to, coughing, coughing up blood, breathing difficulties, loss of appetite, fatigue, recurring infections, bone pain, swelling in face, arms or neck, headaches, dizziness, limbs that become weak or numb, jaundice, and lumps in the neck or collar bone region. Breathing difficulties may include, but are not limited to, shortness of breath, wheezing and noisy breathing.

Treating a subject may comprise an improvement in and/or the elimination of one or more symptoms of pancreatic cancer in the subject. The one or more symptoms of pancreatic cancer may include, but are not limited to, jaundice, digestive problems, pain in the upper abdomen, loss of appetite, nausea, sudden weight loss, swollen gallbladder, blood clots, and diabetes. Digestive problems may include, but are not limited to, abnormal stools, nausea, and vomiting.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A method for treating cancer in a human subject, comprising:
administering a therapeutic regimen comprising a drug or a combination of drugs to a human subject with a cancer that is associated with expression of at least one human mutant KRAS protein variant, wherein
the drug or combination of drugs, when provided to genetically modified Drosophila that express the at least one human mutant KRAS protein variant or an ortholog thereof that promotes tumorigenesis in the Drosophila, then results in the inhibition of tumorigenesis in the Drosophila.

2. The method of claim 1, wherein the at least one human mutant KRAS protein variant is associated with a mutation in at least one of KRAS codon 12, 13, 18, 61, 117, or 146.

3. The method of claim 1, wherein the at least one human mutant KRAS protein variant comprises a mutation in at least one of G12A, G12C, G12D, and/or G12V.

4. The method of claim 1, wherein the genetically modified Drosophila express the at least one human mutant KRAS protein variant or an ortholog thereof upon induction through an external factor.

5. The method of claim 4, wherein expression of the at least one human mutant KRAS protein variant or an ortholog thereof inhibits the Drosophila larva from surviving to pupation.

6. The method of claim 5, wherein the drug or combination of drugs, when given to the Drosophila larva allows the Drosophila larva to survive to pupation.

7. The method of claim 1, wherein the cancer is an adenocarcinoma of the lung and/or colon.

8. The method of claim 2, wherein the external factor is temperature.

9. The method of claim 1, wherein the therapeutic regimen comprises an anti-cancer therapy.

10. The method of claim 9, wherein the anti-cancer therapy is selected from the group consisting of alkylating agents, anthracyclines, cytoskeletal disruptors, histone deacetylase inhibitors, inhibitors of topoisomerase I, inhibitors of topoisomerase II, kinase inhibitors, nucleotide analogs and precursors analogs, peptide antibiotics, platinum-based agents, retinoids, and vina aklaloids, and derivatives thereof.

11. A method for screening a drug or a combination of drugs for the treatment of a cancer associated with expression at least one human mutant KRAS protein variant, comprising:
providing the drug or combination of drugs to genetically modified Drosophila that express the at least one human mutant KRAS protein variant or an ortholog thereof that promotes tumorigenesis in the Drosophila, and
evaluating whether the drug or combination of drugs results in the inhibition of tumorigenesis in the Drosophila.

12. The method of claim 11, wherein the at least one human mutant KRAS protein variant is associated with a mutation in at least one of KRAS codon 12, 13, 18, 61, 117, or 146.

13. The method of claim 11, wherein the at least one human mutant KRAS protein variant comprises a mutation in at least one of G12A, G12C, G12D, and/or G12V.

14. The method of claim 11, wherein the genetically modified Drosophila expresses the at least one human mutant KRAS protein variant or an ortholog thereof upon induction through an external factor, and the method comprises inducing expression of the protein through the external factor.

15. The method of claim 14, wherein expression of the at least one human mutant KRAS protein variant or an ortholog thereof inhibits Drosophila larva from surviving to pupation and wherein providing the drug or combination of drugs comprises providing the drug or combination of drugs to Drosophila larva.

16. The method of claim 15, wherein evaluating whether the drug or combination of drugs results in the inhibition of tumorigenesis in the Drosophila comprises evaluating whether the Drosophila larva survive to pupation.

17. The method of claim 11, wherein the cancer is an adenocarcinoma of the lung and/or colon.

18. The method of claim 12, wherein the external factor is temperature.

19. The method of claim 11, wherein the drug or combination or drugs comprises an anti-cancer drug.

20. The method of claim 19, wherein the anti-cancer drug is selected from the group consisting of alkylating agents, anthracyclines, cytoskeletal disruptors, histone deacetylase inhibitors, inhibitors of topoisomerase I, inhibitors of topoisomerase II, kinase inhibitors, nucleotide analogs and precursors analogs, peptide antibiotics, platinum-based agents, retinoids, and vina aklaloids, and derivatives thereof.
